(19) United States
(12) Patent Application Publication
(10) Pub. No.: US 2021/0346334 A1
(43) Pub. Date: Nov. 11, 2021

(54) NUTRITIONAL CHROMIUM COMPOSITIONS AND METHODS OF USE

(71) Applicant: Metabolic Therapy Inc., Austin, TX (US)

(72) Inventor: Kenneth O. Russell, Austin, TX (US)

(21) Appl. No.: 17/320,212

(22) Filed: May 13, 2021

Related U.S. Application Data
(63) Continuation-in-part of application No. 17/187,193, filed on Feb. 26, 2021.
Application No. 17/187,193 is a continuation-in-part of application No. 16/442,512, filed on Jun. 16, 2019.

(51) Int. Cl. A61K 31/28; A61K 31/155; A61K 31/192; A61K 31/4406; A23L 33/175

(52) U.S. Cl. CPC A61K 31/28; A61K 31/155; A23L 33/175; A61K 31/4406; A61K 31/192

(57) ABSTRACT

The chromium compositions and methods of use for patients with glucose intolerance comprising a chromium complex in combination with at least one amino acid, niacinamide, and salicylic acid.

### CROSS-REFERENCE TO RELATED APPLICATION

Continuation in-part of reissue application Ser. No. 17/187,193, filed on Feb. 26, 2021, which claims priority of application Ser. No. 12/069,505, filed on Feb. 11, 2008, now Patent No. 9,585,898, continuation in-part of application Ser. No. 16/442,512, filed on Jun. 16, 2019, which is continuation-in-part of application Ser. No. 15/925,437, filed on Mar. 19, 2018, now U.S. Pat. No. 10,398,643, which is continuation-in-part of application Ser. No. 15/154,671, filed on May 13, 2016, now U.S. Pat. No. 10,172,847. The disclosure of which is hereby incorporated by reference herein in its entirety.

### BACKGROUND OF THE INVENTION

In vivo and in vitro studies suggested that chromium enhances insulin sensitivity by promoting insulin receptor signaling. Chromium is necessary for regulating carbohydrate, lipid, and protein metabolism. It is a cofactor of chromodulin, a low-molecular weight oligopeptide in the cell cytoplasm that can bind four chromium ions. Chromodulin binding to the subunit of the insulin receptor activates the tyrosine kinase receptor and amplifies insulin signals that stimulate the activity of glucose transporter protein 4, which is the principal mediator of glucose transport across the cell membrane.

Chromium activated glycolytic pathways allow muscle and adipose cells to uptake and convert glucose into triglycerides. The use of chromium (III) in diabetes treatment has been reported (Lewicki et al., 2014), where the absorption efficiency of chromium depends on the amounts and forms of chromium in the diet. Organic chromium such as chromium picolinate, propionate-methionine chromium, and chromium yeast is more efficiently absorbed than inorganic chromium. After chromium is released into the blood, it is bound by proteins and transported to chromodulin in the cells, activating glucose uptake.

Recent study has reported a positive association between chromium and changes in lipid profiles and glycemic control in type 1 diabetes mellitus patients. Chromium concentrations in men were positively correlated with fasting plasma glucose (P. 0.0149) and high-density lipoproteins (HDLs, P. 0.0079). The relationships among serum trace elements, oxidative stress, hemoglobin A1C (HAC), stage growth, and insulin dose in young type 1 diabetes mellitus patients have been evaluated (Lin et al., 2015). Serum chromium concentrations were lower in diabetic patients than in healthy individuals at three growth stages but were not statistically significant.

Chromium represents an important biological trace element that is greatly involved in the protection against diabetes by improving metabolism. Its blood level is lower in type 2 diabetic subjects than in healthy ones (Sreekanth, 2008). The trivalent chromium ion was found to be essential for insulin signaling since it plays an essential role in various enzymatic reactions (Jaishankar et al., 2014). It acts also as a cofactor for insulin by increasing its binding to receptors (Ali A et al., 2011; Vincent, 2004), thereby enhancing insulin receptor phosphorylation (Wang et al., 2005). Chromium enhances glucose transport in muscle and adipose tissues through stimulation of glucose transportor (GLUT4) translocation to the extracellular membrane (Celafu &. Hu, 2004).

Chromium is found in relatively high amounts in whole grains, egg yolks, broccoli, and brewer yeast. The absorption of chromium from diet is low in efficiency (0.4-2.5%). Chromium is transported to the liver and sequestered by transferrin for uptake by spleen, soft tissue, and bone. Most absorbed chromium is excreted in urine, whereas unabsorbed chromium is excreted in feces. No chromium supplementation is needed in healthy persons because they are not chromium deficient (EFSA NDA Panel (EFSA Panel on Dietetic Products, Nutrition and Allergies), 2014. Scientific opinion on dietary reference values for chromium. J EFSA 2014; 12:3845-3870). Currently, there are no reports on chromium supplementation in type 1 diabetic patients. However, studies with mouse models found that chromium supplementation improved glycemic control.

The intestinal absorption of dietary chromium at daily intakes of 40 μg and more is approximately 0.5% of the total amount present. However, intakes of less than 40 μg/day are absorbed with an increasing efficiency, up to about 2% of the total (Anderson and Kozlovsky, 1985)

U.S. Pat. No. 5,948,772 discloses that the combination of chromium and nicotinic/picolinic acid, in effective amount between 50 and about 10,000 μg, facilitates the absorption of monovalent, divalent and trivalent metal ions by transporting them across intestinal cells and into the bloodstream.

Pat. App. No. WO2008126088A2 discloses that the doses of nicotinic acid over 50 mg may cause flushing of the skin, lasting about 60 minutes, along with a mild itching sensation and a reddening of the skin. Nicotinic acid can cause vasodilation of cutaneous blood vessels resulting in increased blood flow, principally in the face, neck and chest. This produces the niacin or nicotinic acid-flush. The niacin-flush is thought to be mediated via the prostaglandin (PG) prostacyclin and via histamine release. When the nicotinic acid is given repeatedly, tolerance to nicotinic acid-induced flushing tolerance within about a week.

Canadian Pat. App. Mo. 2841731 discloses that nicotinamide (IUPAC name pyridine-3-carboxamide), also known as niacinamide and nicotinic acid amide, is the amide of nicotinic acid (vitamin B3/niacin). Nicotinamide is a water-soluble vitamin and is part of the vitamin B group. Nicotinic acid, also known as niacin, is converted to nicotinamide in vivo, and, though the two are identical in their vitamin functions, nicotinamide does not have the same pharmacologic and toxic effects of niacin, which occur incidental to niacin's conversion. Thus, nicotinamide does not reduce cholesterol or cause flushing. Additionally, nicotinamide lacks of the vasodilator, gastrointestinal, hepatic, and hypolipidemic actions of nicotinic acid. As such, nicotinamide has not been shown to produce the flushing, itching, and burning sensations of the skin as is commonly seen when large doses of nicotinic acid are administered orally.

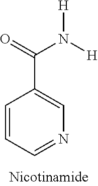

Studies have shown that providing additional supplementation of 3-10g arginine (IUPAC name 2-amino-5-(diaminomethylidene amino) pentanoic acid) three times daily potentially increase vasodilation, circulation, delivery of NAD precursor, and angiogenesis (Penberthy, W. T., 2012). Arginine is well known to promote vasodilation during exercise (Kubota T. et al, 1997) or hypercholestermia. Niacin increases brain endothelialnitric oxide synthase protein expression which is known to cause increased basal vasodilation and angiogenesis (Chen J. et al., 2007). Thus, it would be reasonable to include additional arginine to sustain the vasodilation.

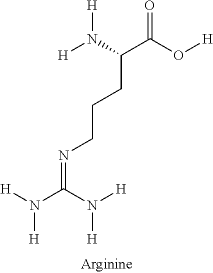 [SMILES: NC(N)=NCCC[C@H](N)C(=O)O]

Salicylic acid (IUPAC name 2-hydroxybenzoic acid) is a vasodilator approved by the FDA for food additive and GRAS. It is safe, inexpensive, biocompatible and easy to produce. It has the following structural formula:

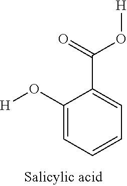

Recent studies who that herb such as Nigella sativa (N. sativa) improves glucose homeostasis and serum lipids in type 2 diabetes. Clinical and statistical significant reduction in FBS and HbA1c levels following N. sativa consumption provides strong evidence for incorporation of N. sativa as part of therapy in diabetes (Daryabeygi-Khotbehsara R., 2017). Mechanisms underlying the therapeutic effects of N. sativa on glycemia suggest amelioration of pancreatic β-cells leading to insulin secretion (Alimohammadi S. et al, 2013 and Kanter M. et al, 2003) reducing hepatic gluconeogenesis (Fararh K. et al, 2004) and inducing insulin sensitivity in peripheral tissue (Benhaddou-Andaloussi A et al, 2004). These effects are attributable to active ingredients of N. sativa (e.g. thymoquinone, dithymoquinone, linoleic acid and oleic acid). Study has shown that thymoquinone treatment on streptozotocin- nicotinamide-induced diabetic rats exerted hypoglycemic benefits (Pari L. et al, 2009). The administration of thymoquinone significantly lowers plasma blood glucose level and increases in insulin level in a dose-dependent manner and protective and insulinotrophic action of thymoquinone on pancreatic β-cells was noticed at higher dose.

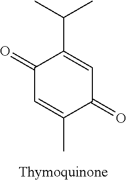

Thymoquinone has been reported to have properties of slow absorption and rapid elimination when administered perorally (Alkharfy K. M. et al, 2015). A nanoformulation of thymoquinone can increase its absorption and bioavailability utilizing natural polymers like chitosan, starch, dextran, carboxymethyl cellulose, albumin, gelatin, alginate, and gums have been widely used. Anionic polymers like gum arabic, gum tragacanth, guar gum, gellan gum and xanthan gum have been also extensively used (Rani R. et al, 2018).

### Definitions

“About” as used herein may refer to approximately a +/−10% variation from the stated value. It is to be understood that such a variation is always included in any given value provided herein, whether specific reference is made to it.

“Pharmaceutically acceptable” means that the agent should be acceptable in the sense of being compatible with the other ingredients of the formulation (as well as non-injurious to the individual). Such excipients include inert diluents such as calcium carbonate, sodium carbonate, lactose, calcium phosphate or sodium phosphate; granulating and disintegrating agents, such as corn starch and alginic acid; binding agents such as starch, gelatin or acacia; and lubricating agents such as magnesium stearate, stearic acid or talc. Tablets may be uncoated or may be coated with known techniques to delay disintegration and absorption in the gastrointestinal tract and thereby provide a sustained action over a longer period of time. For example, a time delay material such as glyceryl monostearate or glyceryl stearate alone or with a wax may be employed.

The term “fatty acid” means a fatty acid that has four (4) to twenty-four (24) carbon atoms.

“Pharmacologically effective amount” means that the concentration of the drug is such that in the composition it results in a therapeutic level of drug delivered over the term that the gel is to be used. Such delivery is dependent on a number of variables including the drug, the form of drug, the time period for which the individual dosage unit is to be used, the flux rate of the drug from the gel, surface area of application site, etc. The amount of drug necessary can be experimentally determined based on the flux rate of the drug through the gel, and through the skin when used with and without enhancers.

“Fixed combination” should be understood as meaning a combination whose active principles are combined at fixed doses in the same vehicle/medium (single formula) that delivers them together to the point of application.

“Treatment” as used herein refers to any treatment of a human condition or disease and includes: (1) preventing the disease or condition from occurring in a subject which may be predisposed to the disease but has not yet been diagnosed as having it, (2)inhibiting the disease or condition, l.e., arresting its development, (3) relieving the disease or condition, i.e., causing regression of the condition, or (4) relieving the conditions caused by the disease, i.e., stopping the symptoms of the disease.

### SUMMARY OF THE INVENTION

The present invention is an improved pharmaceutical composition for supplementing daily nutritional chromium and increasing absorption of trace minerals, while reducing flushing effects. The composition comprising a chromium complex in combination with pyridine-3-carboxamide, 2-amino-5-(diaminomethylidene amino) pentanoic acid, and/or 2-hydroxybenzoic acid. The composition is incorporated into a pharmaceutically acceptable carrier such as tablet, capsule, emulsion, powder, granule, suspension, syrup or elixir.

Chromium complex and niacinamide is in a ratio of between about 1:10 (w/w). The composition further comprises at least one of an amino acid such as 2-amino-5-(diaminomethylidene amino) pentanoic acid for vasodilation to increase absorption of chromium complex and to reduce niconitic acid-induced skin flushing effect.

It is also contemplated by this present invention that the composition contains herb such as Nigella sativa. The hexane extract of the herb contains both long and medium chain fatty acids, and therefore exhibits significant enhancement of intestinal absorption for low permeable chromium.

The present invention also provides a method for supplementing daily nutritional chromium in a human subject by administering chromium complex in combination with niacinamide, arginine, and/or salicylic acid.

The tablet or capsule is extended release of chromium and niacinamide following the administration of arginine. Advantageously, the effective amount of niacinamide is between about 50 and about 10,000 micrograms, and arginine is between about 3 and about 10 grams.

Although the invention has been described with respect to specific embodiments and examples, it should be appreciated that other embodiments utilizing the concept of the present invention are possible without departing from the scope of the invention. The present invention is defined by the claimed elements, and any modifications, variations, or equivalents that fall within the true spirit and scope of the underlying principles.

### BRIEF DESCRIPTION OF THE PREFERRED EMBODIMENTS

In one embodiment, the chromic complex, pyridine-3-carboxamide, and 2-amino-5-(diaminomethylidene amino) pentanoic acid may be incorporated into a tablet, aqueous or oil suspension, dispersible powder or granule, emulsion, hard or soft capsule, syrup or elixir.

In other preferred embodiment, the amino acid may be selected from cysteine, glutamine, glycine, proline, and tyrosine.

In other preferred embodiment, the components of the composition may also be administered separately. Compositions may be prepared in according any method known in the art for the manufacture of pharmaceutically acceptable compositions and such compositions may contain one or more of the following agents: sweeteners, flavoring agents, coloring agents and preservatives. Tablets containing the active ingredients in admixture with non-toxic pharmaceutically acceptable excipients suitable for tablet manufacture are acceptable.

In other preferred embodiment, dispersible powders and granules of the invention suitable for preparation of an aqueous suspension by the addition of water provide the active ingredient in admixture with a dispersing or wetting agent, a suspending agent, and one or more preservatives. Additional excipients, for example sweetening, flavoring and coloring agents, may also be present.

In other preferred embodiment, the dosage range of chromium administered to an individual in the form of chromium/niconitic acid/arginine/salicylic acid, or chromium/niacinamide/arginine provides between about 100 and 1,000 micrograms per day of chromium; preferably between about 200 and 1,000 micrograms per day; more preferably, between about 100 and 500 micrograms per day.

In other preferred embodiment, the ratio of chromium to niacinamide/arginine ranges from about 10:1 to about 1:10 (w/w), more preferably from about 5:1 to about 1:5 (w/w).

It will be appreciated that although specific embodiments of the invention have been described herein for purposes of illustration, various modifications may be made without deviating from the spirit and scope of the invention. Accordingly, the invention is not limited except as by the appended claims.

The compositions are administered in any suitable manner, often with pharmaceutically acceptable carriers. Suitable methods of administering compositions in the context of the present invention to a subject are available, and, although more than one route can be used to administer a particular composition, a particular route can often provide a more immediate and more effective reaction than another route.

The compositions are administered in any suitable manner, often with pharmaceutically acceptable carriers. Suitable methods of administering compositions in the context of the present invention to a subject are available, and, although more than one route can be used to administer a particular composition, a particular route can often provide a more immediate and more effective reaction than another route.

The present invention is further illustrated by the following examples, which should not be construed as limiting in any way. The contents of all cited references throughout this application are hereby expressly incorporated by reference. The practice of the present invention will employ, unless otherwise indicated, conventional techniques of pharmacology and pharmaceutics, which are within the skill of the art.

Therefore, those skilled in the art will appreciate that the conceptions and specific embodiments disclosed in this description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

Throughout this application various publications are referenced. The disclosures of these publications in their entireties are hereby incorporated by reference into this application in order to describe more fully the state of the art to which this invention pertains.

As various modifications could be made to the exemplary embodiments, as described above with reference to the corresponding illustrations, without departing from the scope of the invention, it is intended that all matter contained in the foregoing description and shown in the accompanying drawings shall be interpreted as illustrative rather than limiting. Thus, the breadth and scope of the present invention should not be limited by any of the above described exemplary embodiments, but should be defined only in accordance with the following claims appended hereto and their equivalents.

We claim:

1. A pharmaceutical composition of chromium supplement comprising a chromium complex in combination with pyridine-3-carboxamide, and 2-amino-5-(diaminomethylidene amino) pentanoic acid.

2. The pharmaceutical composition of claim 1 further comprises 2-hydroxybenzoic acid

3. The pharmaceutical composition of claim 1 further comprises a pharmaceutically acceptable carrier, adjuvant, excipient or diluent.

4. The pharmaceutical composition of claim 3, wherein said pharmaceutically acceptable carrier is selected from the group consisting a pill, capsule, lozenge, caplet, syrup, emulsion, suspensional liquid, powder, spray, cream or lotion.

5. The pharmaceutical composition of claim 1, wherein said chromium complex is selected from the group consisting of chromium picolinate, chromium nicotinate, chromium glycinate, or other chromium comprising chromium content of between about 100 to 1,500 micrograms.

6. The pharmaceutical composition of claim 1 is in unit dosage form.

7. A method for increasing absorption of chromium supplement in a human subject, said method comprises administering to said human subject an effective amount of the pharmaceutical composition of claim 1.

8. The method of claim 7, wherein the pharmaceutical composition is administered in combination with a pharmaceutically acceptable carrier.

9. The method of claim 8, wherein the pharmaceutically acceptable carrier is further selected from the group consisting a pill, capsule, lozenge, caplet, syrup, emulsion, suspensional liquid, powder, spray, cream or lotion.

10. The method of claim 7, wherein the pharmaceutical composition is administered orally, nasally, sublingually, intramuscularly, subcultaneously, and transdermally.
